SEQUENCE LISTING (5 sequences)

SEQ ID NO: 1 - protein, 9 aa, Acinetobacter calcoaceticus
FSWGAEGQK

SEQ ID NO: 2 - protein, 9 aa, Acinetobacter calcoaceticus
ISRFAWGEV

SEQ ID NO: 3 - protein, 9 aa, Bovine myelin
LSRFSWGAE

SEQ ID NO: 4 - protein, 9 aa, Agrobacterium tumefaciens
YTRFTWGAP

SEQ ID NO: 5 - protein, 9 aa, Ruminococcus albus
YTQFEISAE

(19) United States
(12) Patent Application Publication
(10) Pub. No.: US 2005/0214862 A1
(43) Pub. Date: Sep. 29, 2005

(54) Diagnosis of spongiform disease

(76) Inventor: Alan Ebringer, London (GB)

Correspondence Address: Joseph T. Leone;DeWitt Ross & Stevens S.C., Suite 401, 8000 Excelsior Drive, Madison, WI, 53717, US

(21) Appl. No.: 10/889,150

(22) Filed: Jul. 12, 2004

Related U.S. Application Data
(63) Continuation-in-part of application No. 09/646,579, filed on Dec. 27, 2000, now Pat. No. 6,849,418.

(30) Foreign Application Priority Data
Mar. 19, 1999 (WO) PCT/GB99/00876
Mar. 19, 1998 (GB) GB 9805913.2
Sep. 29, 1997 (WO) PCT/GB97/02667
Sep. 27, 1996 (GB) 9620195.9

(51) Int. Cl. G01N 33/53; G01N 33/554; G01N 33/569

(52) U.S. Cl. 435/7.1; 435/7.32

(57) ABSTRACT

A diagnostic test is provided for testing for spongiform encephalopathy and other demyelinating conditions in mammals such as BSE in cattle, and MS or CJD in humans, by assaying antibodies present in the mammal which bind to an Acinetobacter species containing an antigenic peptide which exhibits molecular mimicry of a mammalian myelin peptide, e.g. one having the sequence ISRFAWGEV. The test antigen used may be whole Acinetobacter or an antigenic having a sequence present therein, or a prepared or synthetic version of such peptide. The preferred assay is directed to IgA antibodies. Sera of animals or patients having the disease usually register antibody levels at least about two standard deviations above that of healthy control sera.

### CROSS-REFERENCE TO RELATED APPLICATIONS

This is a continuation-in-part of co-pending U.S. application Ser. No. 09/646,579, filed Dec. 27, 2000, (which claims priority to PCT/GB99/00876, filed Mar. 19, 1999, which claims priority to GB 9805913.2, filed Mar. 19, 1998) and of co-pending U.S. application Ser. No. 09/269,607 filed Feb. 20, 2003 (which claims priority to PCT/GB97/02667, filed Sep. 29, 1997, which claims priority to GB 9620195.9, filed Sep. 27, 1996), the contents of all of which are incorporated herein by reference.

### FIELD OF THE INVENTION

This invention relates to the detection of spongiform encephalopathy and other demyelinating conditions in mammals and is particularly, but not exclusively, concerned with the diagnosis of bovine spongiform encephalopathy (BSE).

### BACKGROUND

BSE is a recently discovered and as-yet-poorly characterized neurological disorder of cattle. BSE was first reported in the U.K. after 1982, following a change in the preparation of “bone and meal” feeds. BSE has attracted considerable public health concerns, worldwide, lest it be transmitted to humans following meat consumption. It has been suggested that BSE is caused by “prions,” a type of infectious protein.

### BRIEF DESCRIPTION OF THE DRAWINGS

FIG. 1 is a histogram depicting total Ig antibody titers to Acinetobacter bacteria (solid horizontal line=mean) for 30 control animals aged less than 30 months (A<30 m), 28 controls aged more than 30 months (A>30 m), and 18 controls from the Central Veterinary Laboratory (CVL) as compared to sera from 29 BSE-positive animals, when tested against Acinetobacter calcoaceticus. The broken line represents 95% confidence limits for mean of controls: (A<30 m)+(A>30 m); a one-tailed test. OD=optical density.

FIG. 2 is a histogram depicting IgA antibodies to Acinetobacter bacteria, as measured by the ELISA described in the Examples for various human test populations: C=healthy controls; AS=patients with ankylosing spondylitis; RA=patients with rheumatoid arthritis; CVA=patients with cerebro-vascular accidents; ENC=patients with viral encephalytis; MS=patients with multiple sclerosis; CJD=patients with Creutzfeld-Jakob disease. p-Values indicate significance of results as compared to controls.

FIG. 3 is a histogram depicting IgA antibody titers to Acinetobacter bacteria (solid horizontal line=mean) for 30 control animals aged less than 30 months (A<30 m), 28 controls aged more than 30 months (A>30 m), and 18 controls from the Central Veterinary Laboratory (CVL) as compared to sera from 29 BSE-positive animals, when tested against Acinetobacter calcoaceticus. The broken line represents 95% confidence limits for mean of controls: (A<30 m)+(A>30 m); a one-tailed test. OD=optical density.

FIG. 4 is a histogram depicting total antibody titers to Acinetobacter calcoaceticus bacteria (solid horizontal line=mean) for patients suffering from MS as compared to healthy controls. p-Value indicates significance of the results as compared to controls.

FIG. 5 is a histogram depicting total antibody titers to Acinetobacter calcoaceticus (A11171) bacteria (solid horizontal line=mean) for patients suffering from MS as compared to healthy controls. p-Value indicates significance of the results as compared to controls.

### DETAILED DESCRIPTION OF THE INVENTION

The present invention is based on an alternative model of the genesis of various forms of spongiform encephalopathy and other demyelinating conditions in mammals. According to the proposed model, BSE and related diseases, including Creutzfeld-Jakob's disease (CJD) in humans and multiple sclerosis (MS) in humans are conceived as autoimmune diseases arising as a result of molecular mimicry between certain infective agents and the myelin of the infected mammal. This new model of BSE, in particular, is based on the following experimental observations.

A characteristic histopathological feature of BSE is a “spongiform” appearance, which also occurs in chronic but not acute “experimental allergic encephalomyelitis” (EAE), at least in rabbits and guinea pigs. A short sequence of bovine myelin (FSWGAEGQK) (SEQ. ID. NO: 1), which withstands denaturation following heating to 100° C. for one hour, was reported over twenty-five years ago to produce hind quarters paralysis, tremors and death, following inoculation into guinea pigs, which to some extent resembles the features observed in cattle suffering from BSE. In accordance with the present invention, this sequence has been used as a computer probe to search for proteins showing molecular mimicry. This sequence, in denatured form, may be described as encephalitogenic.

Analysis of proteins in databases (Genbank and SwissProt) revealed that three microbes showed molecular mimicry of the bovine myelin sequence, the best one being found in 4-carboxy-muconolactone-decarboxylase of Acinetobacter calcoaceticus, a common microbe present in soil and water supplies. These sequence similarities are shown in Table 1.

TABLE 1. Comparison of Amino Acids of Bovine Myelin to Microorganisms from Genbank and SwissProt which have Similar Sequences in other Proteins

TABLE 1

| | | Posi- | |
|---|---|---|---|
| Source | Amino acids | tions | Locations |
| Bovine myelin | LSRFSWGAE | 110-118 | 4-carboxy- |
| | (SEQ. ID. NO: 3) | | muconolactone |
| | | | decarboxylase |
| Acinetobacter | ISRFAWGEV | 41-49 | Beta- |
| calcoaceticus | (SEQ. ID. NO: 2) | | glucosidase |
| Agrobacter | YTRFTWGAP | 693-701 | Beta- |
| tumefaciens | (SEQ. ID. NO: 4) | | glucosidase |
| Ruminococcus | YTQFEISAE | 274-282 | |
| albus | (SEQ. ID. NO: 5) | | |

Alphabetic letters refer to biochemical symbols for amino acids.

In conformity with the new model, it has now been found that sera of BSE-affected cattle contain significantly higher levels of antibodies to Acinetobacter species as compared to normal controls. Quite unexpectedly, it has also been found that serum samples of human patients suffering from MS and CJD also contain significantly higher levels of antibodies to Acinetobacter species as compared to normal controls.

The present invention therefore provides a diagnostic test for spongiform encephalopathy and other demyelinating conditions, such as MS and CJD, in mammals which comprises assaying antibodies present in the mammal which bind to an antigenic peptide which exhibits molecular mimicry of a mammalian myelin peptide, especially one having the sequence FSWGAEGQK (SEQ. ID. NO: 1) or ISRFAWGEV (SEQ. ID. NO: 2). The term “molecular mimicry” refers to a degree of similarity (sequence homology) as between the antigenic peptide and a myelin peptide which results in the formation of antibodies which cross-react with myelin and demyelinate nervous tissue. The presence of such antibodies at elevated levels compared to those found in unaffected animals is therefore a marker for BSE which may be used to detect BSE at an early stage at which curative or other appropriate action may be taken.

The assay may be carried out using the whole Acinetobacter or other organism as the test antigen. Any strain of Acinetobacter having an antigenic peptide identified above may be used. Alternatively the isolated peptide or a synthetic form of the peptide may be used as antigen. Any suitable type of assay procedure may be used, the ELISA method being especially convenient.

Antibody levels indicative of BSE, MS, and CJD are those which are significantly higher than the control levels. Usually, levels elevated to about 2 standard deviations above the controls may be taken as a positive indication but margins around this figure may be possible or desirable for purposes of caution.

Procedures for carrying out an assay in accordance with this invention in order to detect Bovine spongiform encephalopathy (BSE) in cows, or Creutzfeld-Jakob's disease (CJD) or Multiple sclerosis (MS) in humans, are described in the following illustrative Examples, the first of which compares sera from animals known to have had BSE with sera from healthy animals.

A test kit for use according to the invention therefore contains at least one test antigen corresponding to an Acinetobacter epitope as indicated. In order to reveal IgA antibodies the kit also contains a secondary antibody against the human, bovine, or other mammalian IgA, if the ELISA protocol is used.

### EXAMPLE 1

Assay of Total Ig Antibodies in Bovines Materials and Methods

Bovine Sera: Sera from 29 animals, which were found at post-mortem to satisfy the criteria of BSE and 18 animals which did not, were supplied by the Central Veterinary Laboratory (CVL) (New Haw, Addlestone, Surrey), an executive agency of the U.K. Ministry of Agriculture, Fisheries and Food (MAFF). The 18 animals which did not have BSE had been referred to CVL because of abnormal behaviour but post-mortem examinations carried out by MAFF had excluded BSE.

Furthermore, 30 sera from animals aged less than 30 months (A<30 M) (8 Friesians, 21 Hereford-Friesian and 1 Charolais-Friesian crossbreeds) and 28 sera from animals aged more than 30 months (A>30 M) (all dairy Friesians), were used as further controls. These were collected from a farm, kept under “organic farming” conditions where no case of BSE had been reported. Serum samples were obtained during routine herd testing.

Preparation of Bacteria: Acinetobacter calcoaceticus was obtained from the National Collection of Industrial and Marine Bacteria Ltd. NCIMB 10694 (Aberdeen). Cultures were grown in 21 flasks on an orbital shaker for 2 days at 30° C., in 200 ml nutrient broth (Oxoid; 25 g/1). Flasks were inoculated with 10 ml of the corresponding starter culture left shaking at 37° C. for 6 hours. Batch culture cells were harvested by centrifugation at 6000 r.p.m. for 20 minutes at 4° C. (MSE 18.6×250 ml rotor). The pellets of cells were then washed three times with 0.15 M phosphate-buffered saline (PBS; pH 7.4) before being finally resuspended in 20 ml of PBS. A stock solution of the suspension was prepared by diluting in 0.05 M carbonate buffer (pH 9.6) to give an optical density (OD) reading of 0.25 on the spectrophotometer (Corning Model 258).

Enzyme-Linked Immunosorbent Assay: ELISA assays were carried out in the conventional manner. Briefly ELISA plates were coated with bacteria overnight at 4 C and the non-specific sites blocked with PBS containing 0.1% Tween, 0.2% ovalbumin (Sigma, Grade III), plates washed and a 1/200 dilution of test or control serum added. The plates were incubated at 37 C for 1 hour, washed and rabbit anti-cow immunoglobulin (IgG+IgA+IgM) (1:4000) (Dako Ltd.) added. The plates were reincubated for 2 hours, washed and substrate added. The reaction was stopped with a 2 mg/ml solution of sodium fluoride (Sigma). The plates were read at 630 nm on a microtitre plate reader (Dynatech MR 600) and results expressed as OD±S.E. All studies were carried out under code in that the tester did not know which were test or control sera. The mean OD units of total immunoglobulin antibodies in different groups were compared using Student's t-test.

ELISA Protocol:

1. Dilute antigen in coating buffer, add 200 l to each well. Incubate overnight at 4° C. wrapped in foil.

2. Wash out the antigen, using washing/incubation buffer; the wells of the tray should be completely full during the washing stages as the Tween-20 prevents any further protein from being absorbed onto the plastic. Wash 3 times, leaving for approx. 4 minute intervals at room temperature.

3. Incubate the plate at 37° C. for 1 hr with 0.2% Ovalbumin in washing/incubation buffer.

4. Add 200 l of test serum. Dilutions are made in washing/incubation buffer. Incubate for 2 hours at 37° C. wrapped in foil.

5. Repeat washing process as in 2.

6. Add 200 l Horseradish peroxide HRP-conjugated second antibody, also diluted in washing/incubation buffer.

7. Repeat washing process as in 2.

8. Add 200 l substrate (ABTS) to wells; leave to develop colour for approx. 20 minutes in the dark at room temperature. Stop reaction with 100 l of stopping solution and read plate at 630 nm.

Results:

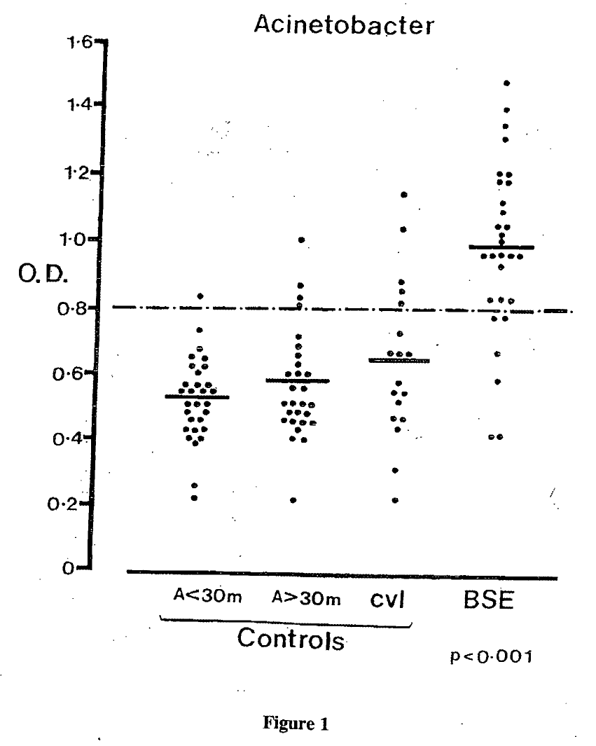

Antibodies to A. calcoaceticus of total immunoglobulin (IgG+IgA+IgM) were significantly elevated in the BSE sera (mean±SE: 0.99±0.05) when compared to CVL controls (0.65±0.06) (t=4.48, p<0.001), organic farming controls aged more than 30 months (0.57±0.03) (t=7.19, p<0.001), and organic farming controls aged less than 30 months (0.53±0.02) (t=8.64, p<0.001). These results are shown in FIG. 1. In FIG. 1, the antibody titers against Acinetobacter calcoaceticus (bar=mean) for 30 control animals aged less than 30 months (A<30 m), 28 control animals aged more than 30 months (A>30 m), and 18 control animals from the Central Veterinary Laboratory (CVL) are compared to corresponding titers from 29 BSE-infected animals. The dashed line represents 95% confidence limits for the mean of the controls.

As shown in FIG. 1, there was no significant difference between the CVL controls and the organic farming controls aged more than 30 months, but there was a small, statistically significant difference with the sera from animals aged less than 30 months (t=2.41, p<0.05). A re-examination of the CVL control serum with the highest anti-Acinetobacter level of 1.16 OD, showed that it came from a clinically normal control animal, diagnosed as negative to BSE on the statutory diagnostic criteria, and it was also negative when tested for scrapie associated fibrils. This control animal did, however, have white matter vacuolation of the substantia nigra and internal capsule, although this had been seen before and not considered significant.

One clear result from these studies, is that in at least in one “transmissible spongiform encephalopathy” (TSE), namely BSE, a specific immune response can be demonstrated against a microbe that is found readily in the environment of cattle and which also happens to possess a molecular sequence resembling bovine myelin.

### EXAMPLE 2

### Test for IgA Antibodies

Using the bovine sera described in Example 1, the sera was also tested for IgA antibodies. The ELISA used in this Example was prepared as follows herein is as follows:

ELISA Protocol:

1) Aliquots of 200 ul of the diluted suspension of Acinetobacter calcoaceticus (NCIEMB 10694, Aberdeen) grown in nutrient broth are absorbed onto 96 well flat bottomed rigid polystyrene microtitre plates overnight at 4° C.

2) The plates are then washed 3 times with phosphate buffered saline (PBS), 0.1% (v/v) Tween 20.

3) Aliquots of 200 μl of blocking solution (0.2% w/v ovalbumin, 0.1% v/v Tween 200 in PBS is added to each well and incubated for one hour at 37° C.

4) The plates are then washed 3 times with PBS.Tween 20.

5) Aliquots of 200 μl serum samples (test or control) diluted 1/200 in PBS. Tween 20 is added and incubated for 2 hours at 37° C.

6) The plates are then washed 3 times with PBS.Tween 20.

7) Aliquots of 200 μl of peroxidase conjugated rabbit anti-human IgA or rabbit anti-cow IgA, diluted 1/4000 (cow) (or 1/500 for human) with PBS.Tween 20 are added and incubated for 2 hours at 37° C.

8) The plates are then washed 3 times with PBS.Tween 20.

9) The development of the colorimetric assay takes place at room temperature for 20 minutes, after the addition of 200 μl per well of 0.5 mg/ml (2,2′-azinobis(3-ethylbenz-thiazoline-6-sulphonic acid) in citrate/phosphate buffer, pH 4.1, containing 0.98 mM hydrogen peroxide.

10) the reaction is then stopped with 100 μl of 2 mg/ml sodium fluoride and optical densities measured at a wavelength of 630 nm with a micro-ELISA plate reader.

Additionally, the following protocols were performed. These experiments were conducted to show that an elevated level of antibodies specific to Acinetobacter spp. in human subjects, as compared to the corresponding antibody level in a control population of normal human subjects is indicative of MS or CJD in the human subject tested.

Sera from 26 multiple sclerosis (MS) patients (the human test subjects) were obtained. Diagnosis of MS was made according to the Poser criteria, a widely accepted means of diagnosing MS. Sera from 25 normal human subjects (the control group) were also obtained.

Cultures of Acinetobacter sp. strain 11171, Acinetobacter sp. strain 19004, Acinetobacter junil 17908, Acinetobacter lwoffil 5866, and Acinetobacter radioresistens (sp. 12) were obtained from the Public Health Laboratories, Nottingham, UK. Acinetobacter calcoaceticus (NCIMB 16904) was obtained from the National Collections of Industrial and Murine Bacteria, Ltd., Aberdeen, Scotland. The cultures were grown in 1 L flasks on an orbital shaker for 2 days at 30° C., in 200 mL of nutrient broth. Flasks were inoculated with two loopfuls of starter culture and left shaking for 6 hours at 37° C. Batch cultures were harvested by centrifugation. Pellets of cells were washed three times in 0.15 M PBS (pH 7.4) and resuspended in 10 mL of PBS. For the ELISA's described herein, a stock solution of each bacterial suspension was prepared by diluting the suspension in 0.05 carbonate buffer (pH 9.6) to give an OD reading of 0.25 at 540 nm. For SDS-PAGE, the resuspended pellet was ultrasonicated at an amplitude of 12 μm with 30-second bursts and 60-second rest periods (7 cycles). The protein content of the sonicated samples was measured using Bradford's protein assay. The samples were then diluted in sample buffer (0.0625 M Tris-HCl (pH 6.8), 2% SDS, 10% glycerol, 0.001% bromophenol blue) to a protein concentration of 1 μg/μL and heated at 100° C. for 3 minutes.

Enzyme-linked immunosorbent assays (ELISAs) were prepared as noted above and also by taking aliquots of 200 μL of bacterial suspension, or bovine myelin basic protein (MBP), or neurofilaments (25 μ/mL, Sigma), diluted in a 0.05 M carbonate buffer (pH 9.6), and adsorbing these onto a 96-well flat-bottom polystyrene microELISA plate (Dynatech) overnight at 4° C. Plates were washed three times for 5 min. in PBS containing 0.05% wt/vol Tween 20 (“washing” and “incubation” buffer) and were then blocked with PBS containing 0.1% BSA for 1 hr at 37° C. The washing procedure was repeated and 200 μL of control or test serum, diluted 1 in 200 in incubation buffer, was added to the wells in duplicate and incubated for 1 hr at 37° C. Plates were washed three times in washing buffer, and 200 μL of IgM, IgG, or IgA rabbit anti-human conjugate with horseradish peroxidase (diluted 1 in 500 with incubation buffer) was added and incubated for 1 hr at 37° C. The washing procedure was repeated, and 200 μL of substrate solution was added (substrate solution=0.5 mg/mL 2,2′-azino-bis(3-ethylbenzthiazoline-6-sulfonic acid) in citrate phosphate buffer, pH 4.1, containing 0.98 mMH202). The plates were developed in the dark, at room temperature, for 25 min. The reactions were stopped by adding 100 μL of sodium fluoride (2 mg/mL). Absorbances were measured on a microtiter plate reader (Dynatech MR606) at 630 nm. All studies were carried out under blind conditions where the tester did not know which sera were test samples and which sera were controls.

The mean OD units of control groups were compared with the mean OD of the 26 MS patients using a one-tail Student's t-test, and 95% confidence limits of control groups were calculated. Pearson's correlation coefficient (r) was also calculated using the statistical package Prism 3.0 (GraphPad Software).

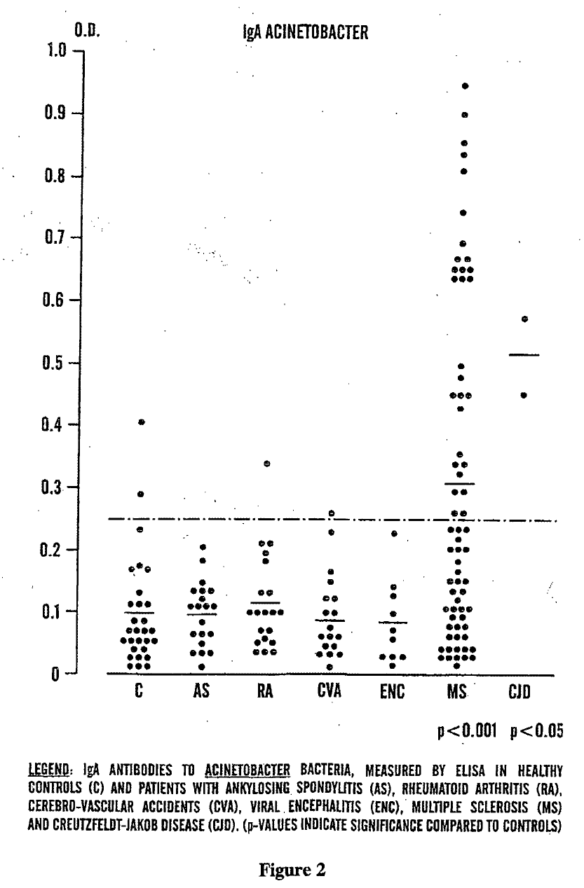

The same protocol was repeated for sample populations of human patients suffering from ankylosing spondylitis (AS), rheumatoid arthritis (RA), cerebro-vascular accident (CVA), viral encephalytis (ENC); multiple sclerosis (MS); Creutzfeld-Jakob disease (CJD). (See FIG. 2.)

The results of the ELISAs described in the immediately preceding paragraphs were as follows:

Levels of IgA antbodies to Acinetobacter sp. strain 11171 (P<0.0001), Acinetobacter sp. strain 19004 (P<0.0001), Acinetobacter junil 17908 (P<0.01), Acinetobacter lwoffil 5866 (P<0.0001), and Acinetobacter radioresistens (P<0.0001) in MS patients were significantly higher than those in the healthy control group.

Levels of IgG antbodies to Acinetobacter sp. strain 11171 (P<0.0001), Acinetobacter sp. strain 19004 (P<0.0001), Acinetobacter junil 17908 (P<0.0001), Acinetobacter lwoffil 5866 (P<0.0001), and Acinetobacter radioresistens (P<0.0001) in MS patients were significantly higher than those in the healthy control group.

Levels of IgM antbodies to Acinetobacter sp. strain 11171 (P<0.0001), Acinetobacter sp. strain 19004 (P<0.0001), Acinetobacter junil 17908 (P<0.0001), Acinetobacter lwoffil 5866 (P<0.0001), and Acinetobacter radioresistens (P<0.0001) in MS patients were significantly higher than those in the healthy control group.

The results for patient populations suffering from MS and CJD are shown in FIG. 2. The p values for both MS and CJD show statistically significant increases in anti-Acinetobacter IgA levels in the MS patient population and the CJD patient population, as compared to normal controls (C) and for patient populations suffering from ankylosing spondylitis (AS), rheumatoid arthritis (RA), cerebro-vascular accident (CVA), and viral encephalytis (ENC)

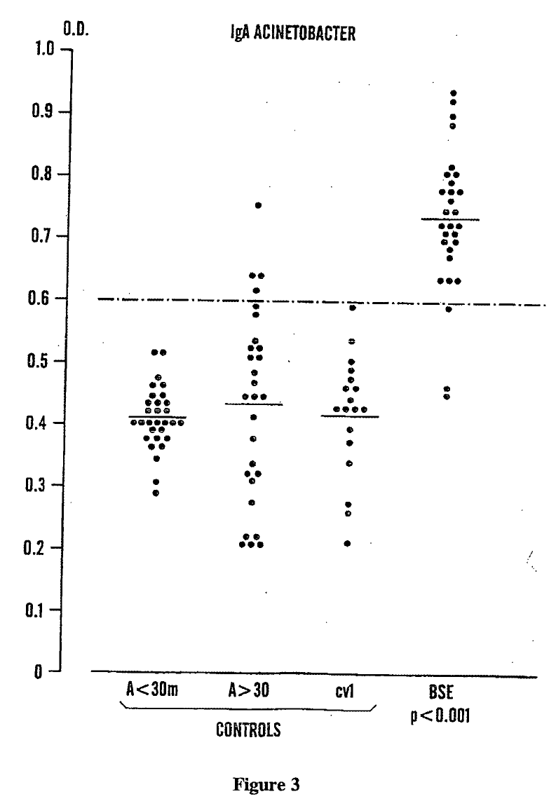

Results for anti-Acinetobacter IgA levels for BSE-infected bovines as compared to the corresponding levels in normal bovines are shown in FIG. 3. (The abbreviations are the same as for FIG. 1.) This figure quite clearly shows that BSE-infected cattle have statistically significant (p<0.001) increases in the level of anti-Acinetobacter IgA in their sera as compared to normal controls.

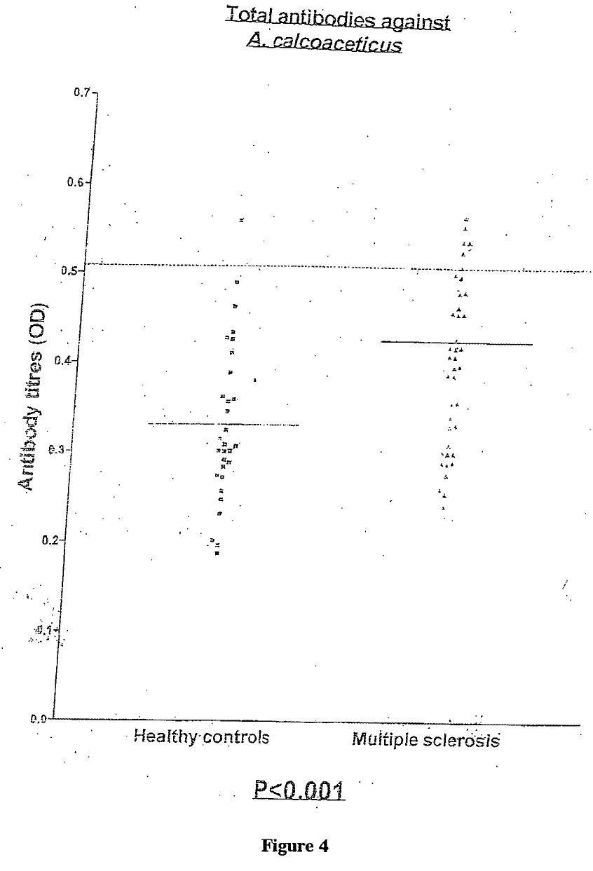

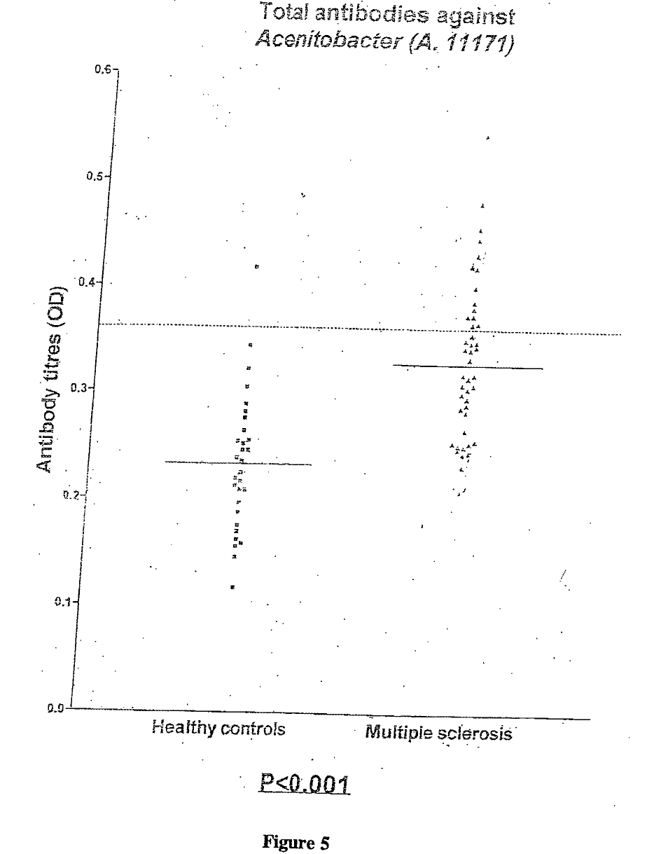

The corresponding phenomenon has also been demonstrated for humans suffering from MS, using as the measure either total Ig antibodies against Acinetobacter calcoaceticus (FIG. 4) and total Ig antibodies against a different species of Acinetobacter (A. 11171) (FIG. 5). The data for these figures was generated using the protocols disclosed in this Example.

In FIG. 4, a test population of human MS patients had their sera tested for total antibodies reactive against A. calcoaceticus. The MS population had a significantly increased level of these antibodies as compared to normal controls.

In the same fashion, in FIG. 5, a test population of human MS patients had heir sera tested for total antibodies reactive against Acinetobacter (A. 11171). The MS population had a significantly increased level of these antibodies as compared to normal controls.

In both of FIGS. 4 and 5 the short, solid horizontal line represents the mean value for each population tested. The long, broken horizontal line represents the 95% confidence limits for the mean of the controls

The significance of these results is that other forms of spongiform encephalopathy, including Creuzfeld Jacob disease (CJD) and Multiple Sclerosis (MS) in humans can be explained and detected on the same model as indicated for BSE. An elevated level of these antibodies in a human subject indicates that the subject is suffering from MS or CJD. Application of the test protocol described above to human CJD sera and MS sera can therefore confirm the presence of cross-reacting antibodies.

Specifically, the present inventor has confirmed the presence of elevated levels of certain antibodies in human sera of patients suffering from multiple sclerosis MS or CJD. Total Ig levels can be measured, or only IgA levels can be measured. Both show statistically-significant increases. These levels are particularly marked for IgA antibodies to Acinetobacter species e.g. Acinetobacter calcoaceticus, the same organisms for which antibodies were found in BSE sera.

The same results (as shown in Example 2) have been obtained for patients suffering from MS and Creutzfeldt-Jakob disease (CJD). Tests for antibodies in sera from patients who had died of CJD also show increased levels of total anti-Acinetobacter Ig, and an especially marked elevation of the IgA antibody sub-class.

It is clear that humans suffering from MS and CJD and cows suffering from BSE all have very significantly raised levels of Acinetobacter calcoaceticus Ig antibodies in their blood. Tests for such antibodies in sera from living subjects at an early stage make it possible to identify those liable to develop these diseases. The present invention opens up the opportunity of early treatment of these infections, for example, by use of an appropriate antibiotic to prevent further auto-immune attack on the subjects' own myelin.

Acinetobacter calcoaceticus is one species of Acinetobacter that provides an antigen which stimulates the formation of antibodies which cross-react with the mammalian myelin . Antibodies have been demonstrated to react with several strains of this species including 17905, AC606, SP13TV, 105/85, and 11171 (see FIG. 5).

In carrying out the present invention, the test is for antibodies which bind to an epitope present in or derived from the Acinetobacter species. The antigen used in the test may be the whole organism or at least one prepared peptide sequence corresponding to an Acinetobacter epitope. Alternatively, peptide sequences may be used which have minor variations in amino-acid sequence from the above-mentioned epitopes or prepared peptides but are conformationally sufficiently similar to them that they also bind to the relevant antibodies. For example, peptides having the sequence RFSAWGAE (SEQ. ID. NO: 1) or ISRFAWGEV (SEQ. ID. NO: 2) may be used.

As indicated above, antibodies are assayed and a positive result is indicated by levels of antibodies at least about two standard deviations above that of control samples.

In view of the greater specificity of the IgA antibodies in the immune response it may be concluded that the mechanism of infection with Acinetobacter is via the mucous membranes of the body, the primary sites being the gut or the nasal passages. Since a further correlation has been observed between MS sufferers and patients with major sinus infections, it is probable that the nasal passages are the site of infection, resulting from inhalation of dust formed from dried sewage or animal excrement and carrying Acinetobacter. The knowledge of this mechanism implies the need for improved hygiene practices.

1. A method for detecting multiple sclerosis (MS), Creutzfeldt-Jakob disease (CJD), or spongiform encephalopathy in a mammal, the method comprising testing a biological sample obtained from the mammal for antibodies which bind to Acinetobacter species.

2. The method according to claim 1, in which the biological sample is tested for antibodies which bind to an Acinetobacter species that presents to the mammal an antigen exhibiting molecular mimicry with myelin of the mammal.

3. The method according to claim 1, in which the antibodies are indicative of prior exposure to Acinetobacter calcoaceticus.

4. The method according to claim 1, in which the disease tested for is bovine spongiform encephalopathy.

5. The method according to claim 1, in which the disease tested for is multiple sclerosis in humans.

6. The method according to claim 1, in which the disease tested for is Creutzfeldt-Jacob disease in humans.

7. The method according to claim 1, wherein a positive result is indicated by levels of antibodies at least about two standard deviations above that of control samples.

8. The method according to claim 1, in which the biological sample is tested for antibodies which bind to an antigen containing the peptide sequence ISRFAWGEV (SEQ. ID. NO: 2).

9. The method according to claim 1, in which the biological sample is tested for antibodies which bind to an antigen containing the peptide sequence RFSAWGAE (SEQ. ID. NO: 1).

10. The method according to claim 1 in which the biological sample is tested for antibodies which bind to whole bacteria of the Acinetobacter species.

11. The method according to claim 1, in which the antibodies tested for are antibodies which bind to an antigen present in or derived from the Acinetobacter species or to a prepared peptide sequence corresponding thereto.

12. The method according to claim 11, in which the antibodies tested for are IgA antibodies.

13. The method according to claim 1, in which the antibodies tested for are antibodies which bind to a peptide sequence that has sufficient conformational similarity to an Acinetobacter antigen such that the antibodies tested for are cross-reactive with the Acinetobacter antigen.

14. A test kit for detecting multiple sclerosis, Creutzfeld-Jakob disease, or spongiform encephalopathy in mammals, the test kit comprising a test antigen, wherein the test antigen is whole Acinetobacter organism or at least one prepared peptide sequence corresponding to an Acinetobacter antigen, and wherein the test antigen is disposed in a suitable container.

15. The test kit according to claim 14, further comprising a secondary antibody against human, bovine, or other mammalian immunoglobulin.

16. The test kit according to claim 14, wherein the test kit comprises an enzyme-linked immunosorbent assay (ELISA) kit.

17. The test kit according to claim 14, comprising a peptide having the sequence RFSAWGAE (SEQ. ID. NO: 1) or ISRFAWGEV (SEQ. ID. NO: 2).

18. The test kit according to claim 14, in which the test antigen is a peptide sequence which is conformationally sufficiently similar to an Acinetobacter antigen to bind to antibodies that bind to the Acinetobacter antigen, the test kit including a secondary antibody against human, bovine, or other mammalian IgA.

19. The test kit according to claim 18, in which the secondary antibody is a rabbit anti-human IgA or rabbit anti-bovine IgA.
